(19) United States
(12) Patent Application Publication
(10) Pub. No.: US 2010/0062418 A1
(43) Pub. Date: Mar. 11, 2010

(54) INACTIVATED AND DRIED BIOLOGICAL PREPARATIONS

(76) Inventors: Patrick A. Mach, Shorewood, MN (US); Tera M. Nordby, Woodbury, MN (US); Mara S. Reif-Wenner, Red Wing, MN (US)

Correspondence Address: 3M INNOVATIVE PROPERTIES COMPANY, PO BOX 33427, ST. PAUL, MN, 55133-3427, US

(21) Appl. No.: 12/515,746

(22) PCT Filed: Nov. 15, 2007
(86) PCT No.: PCT/US07/84738

§ 371 Date: Nov. 13, 2009

Related U.S. Application Data
(60) Provisional application No. 60/867,020, filed on Nov. 22, 2006.

(51) Int. Cl. C12Q 1/70; C12Q 1/02; G01N 33/48; C12N 7/00; C12N 1/36; C12N 1/20; G01N 1/28

(52) U.S. Cl. 435/5; 435/29; 435/40.5; 435/235.1; 435/245; 435/253.4

(57) ABSTRACT

In one aspect, the present invention provides biological sample that includes a dry preparation comprising a stabilizer and stabilized, inactivated biological material. In certain embodiments, the biological material may be heat inactivated and/or heat dried. In another aspect, the present invention provides a method of making a dried biological preparation. Generally, the method includes collecting a sample of biological material, inactivating the biological material, suspending the sample in a volume of a stabilizer and allowing the stabilizer to dry. In certain embodiments, the biological material may be heat inactivated and/or heat dried.

### CROSS REFERENCE TO RELATED APPLICATION

This application claims the benefit of U.S. Provisional Patent Application No. 60/867,020, filed Nov. 22, 2006, the disclosure of which is incorporated by reference herein in its entirety.

### BACKGROUND

Assays that can detect the presence of biological hazards such as, for example, infectious agents, are important tools for monitoring the safety of our environment and the health of individuals. Detection assays may be performed on samples gathered from physiological fluids, process streams, water, soil, plants or other vegetation, air, surfaces (e.g., contaminated surfaces), medicines, and the like.

A negative result obtained by performing a detection assay can provide reassurance that the environment and/or products that are ingested or otherwise introduced into our bodies are safe or that an individual or population is healthy. That reassurance is warranted only when one can be certain that the assay is performing as it should—i.e., that the negative result indicates that the target is not present rather than indicating that the assay failed to detect a target that was, in fact, present.

Many detection assays are designed to detect live cells or viruses or targetcomponents of cells or viruses (e.g., DNA, RNA, or intracellular, extra-cellular, or cell-associated components). Consequently, a positive control often requires a significant amount of sample preparation in order to extract the target components that can be used in the positive control. In other cases, the detection assay may be designed to detect a whole cell, a virus, or some surface component of a cell or virus. In such cases, the positive control requires a fresh sample of the live target. In either case, it may be difficult to perform the positive control outside of a laboratory setting in which the sample preparation and/or cell culture resources are available.

A positive control that can be performed in the absence of sample preparation and/or cell culture resources can provide certain benefits such as, for example, decreasing cost, decreasing assay time, increasing privacy (e.g., in-home tests), increasing reproducibility and reliability, increasing shelf-life, ease-of-use, and, if the positive control material is inactivated, decreasing potentially biohazardous waste. A need exists for a detection assay positive control that can be performed outside of a laboratory setting.

### SUMMARY

In one aspect, the present invention provides biological sample that includes a dry preparation comprising a stabilizer and stabilized, inactivated biological material. In certain embodiments, the biological material may be heat inactivated and/or heat dried.

In another aspect, the present invention provides a method of making a dried biological preparation. Generally, the method includes collecting a sample of biological material, inactivating the biological material, suspending the sample in a volume of a stabilizer and allowing the stabilizer to dry. In certain embodiments, the biological material may be heat inactivated and/or heat dried.

In another aspect, the present invention provides a method of performing a positive control in an assay for detecting target biological material. Generally, the method includes providing a dry preparation that comprises a stabilizer and stabilized, inactivated target biological material, contacting the inactivated target biological material with an aqueous solution, thereby generating a reconstituted sample, and performing the assay for detecting the target biological material on the reconstituted sample. In certain embodiments, the biological material may be heat inactivated and/or heat dried.

Various other features and advantages of the present invention should become readily apparent with reference to the following detailed description, examples, claims and appended drawings. In several places throughout the specification, guidance is provided through lists of examples. In each instance, the recited list serves only as a representative group and should not be interpreted as an exclusive list.

### DETAILED DESCRIPTION OF ILLUSTRATIVE EMBODIMENTS OF THE INVENTION

The present invention provides a positive control for a detection assay that does not require laboratory resources. As a result, the positive control may provide one or more of the following benefits: it can reduce the time, cost, and/or resources required to perform the positive control; it can reduce the time required to determine whether the assay is functioning properly when the sample assay provides a negative result; it can provide a more stable preparation; it can reduce variability, thereby providing more reproducibility and reliability; and the assay may be designed for use by non-technical personnel in a non-technical setting (i.e., in-home testing).

As used herein, the following terms shall have the indicated meanings:

“Biological material” and variations thereof refer to whole cells, cell fragments, cell components, virus particles, and/or fragments of virus particles. When referring to whole cells or cell fragments, the cell in question may be prokaryotic or eukaryotic and, if eukaryotic, may be a eukaryotic single-celled organism or a whole cell from a multicellular organism.

“Dry” and variations thereof refer to substances that are rehydratable.

“Inactivated” refers to a sample that contains essentially no living components (e.g., cells or viruses) capable of propagation.

“Stabilized” and variations thereof refer to biological specimens that, while being inactivated, retain their enzymatic and chemical susceptibilities and retain the reactivity of receptors and recognition elements (for example, antigens, nucleic acid sequences, etc.) of living cells.

As used herein, “a,” “an,” “the,” “at least one,” and “one or more” are used interchangeably. Thus, for example, a biological sample comprising “a” target biological material can be interpreted to mean that the sample includes at least one, and perhaps more than one, target biological material.

Also herein, the recitations of numerical ranges by endpoints include all numbers subsumed within that range (e.g., 1 to 5 includes 1, 1.5, 2, 2.75, 3, 3.80, 4, 5, etc.).

Unless otherwise indicated, reference to a compound can include the compound in any form, including any isomer (e.g., diastereomer or enantiomer), salt, solvate, polymorph, and the like. In particular, if a compound is optically active, reference to the compound can include each of the compound's enantiomers as well as racemic and scalemic mixtures of the enantiomers.

In one aspect, the invention provides a biological preparation that can be used as a positive control for a detection assay. Generally, the biological sample includes a dry preparation that includes a stabilizer and stabilized, inactivated biological material. Because the sample is generally dry, it is light, portable, convenient, and neat. Because the sample is inactivated, the biological material is non-viable—i.e., it cannot be propagated or grown—and it is not infectious. Thus, the sample raises fewer biohazard concerns than if it included living cells or viruses. Because the sample is stabilized, however, it can react in the detection assay as if it were living—i.e., surface components are available for recognition and/or chemical or enzymatic reactions, it can be opened to make internal components available for detection, and the native conformation of recognition elements can be maintained. Thus, the sample can function in the detection assay as if it were living biological material.

In another aspect, the invention provides a method of making a dried biological preparation. Generally, the method includes harvesting a sample of biological material, inactivating the biological material, suspending the sample in a volume of a stabilizer, and allowing the biological material with the stabilizer to dry.

The sample can be biological material of any species for which a detection assay is designed. Exemplary species include pathogenic bacteria, fungi, viruses, and the like. Additional examples can include cells characteristic of certain tumors such as, for example, tumors of the breast, prostate, blood, lymph, and the like.

The biological material in the sample can be any suitable cell, cell fragment, virus, or fragment of a viral particle of the organism that the detection assay is designed to detect.

Suitable biological materials include, for example, whole cells, cell fragments, cell membrane fragments and components, cell wall components, cell surface components, intracellular components (e.g., proteins, DNA, RNA, etc.), virus particles, capsid proteins, protomers, viral envelope components, nucleocapsid components, viral nucleic acids, and the like. The particular biological material used as a positive control for a particular assay should provide a positive response when subjected to analysis using the assay.

For example, in one embodiment, a detection assay may be designed to detect Staphylococcus aureus. Many detectable S. aureus targets exist, any of which may be suitable biological material. Such exemplary targets include, for example, cell-wall proteins such as protein A, penicillin-binding proteins, such as PBP2a or PBP2′ or other cell wall proteins, including cell surface components recognizing adhesive matrix molecules (MSCRAMMs, e.g., fibrinogen-binding proteins (e.g., clumping factors), fibronectin-binding proteins, collagen-binding proteins, heparin/heparin-related polysaccharides binding proteins), capsular polysaccharides and cell-wall carbohydrates (e.g., teichoic acid and lipoteichoic acid); and/or intracellular or extracellular components such as certain membrane components, including lipopolysaccharides. Examples of membrane proteins include for example, cytoplasmic membrane proteins, outer membrane proteins, pilus, flagellar, cilia proteins or components, and cell membrane proteins.

The biological sample may be substantially homogeneous or, alternatively, may include a heterogeneous mixture of components. For example, the biological sample may include biological material from two separate species if, for example, the detection assay is designed to detect the either/or presence of two target species. Alternatively, the heterogeneous mixture can contain two different biological materials from the same species. For example, a sample may include two different biological materials from one target species and may therefore increase the sensitivity of the assay by providing two different targets capable of providing a positive result. In one embodiment, a heterogeneous mixture can contain an intracellular component and a cell surface component. The positive control can reflect this two-target detection strategy by including biological material that can include both detection targets.

The biological material in the sample is inactivated and is, therefore, non-viable. Thus, the biological material cannot propagate from the sample into the environment and, therefore, poses less of a biological hazard risk than sample material that is not inactivated.

A sample may be identified as inactivated if, for example, after performing an inactivation step on the sample, multiple test aliquots from the sample are transferred to a suitable growth medium and incubated under suitable growth conditions. If no growth is observed after a suitable period of time (e.g., 24 hours), then the sample can be considered inactivated. For example, to confirm inactivation of a S. aureus sample, ten 100 μL aliquots may be transferred in parallel to ten tubes, each containing 1 mL of Tryptic Soy Broth (TSB) and incubated at 37° C.±1° C. for 24-30 hours. If none of the test aliquots have grown in the TBS after the incubation period, the S. aureus sample is inactivated.

Biological material may be inactivated by any suitable method that renders the material non-viable but allows the material to remain stabilized. Such methods include heating, drying (e.g., air drying, vacuum drying, etc.), exposing the biological material to UV irradiation, limited autoclaving, certain chemical treatments (e.g., azide, metals, enzymes, limited antibiotics), pressure treatments, mechanical treatment such as sonication, and ultrasonic treatments. In one example, the biological material can be inactivated by heating in a boiling water bath. If desired, more than one of the inactivation methods may be combined.

In some embodiments, the biological sample can be a dry (i.e., rehydratable) preparation in the form of, for example, a powder or a tablet. In some embodiments, the dry preparation may form a dry coating disposed on at least a portion of a coatable surface of a substrate. Preferably, the preparation is heat-dried. Heat drying can be performed as a continuous process whereas, for example, freeze drying or lyophilizing is a batch process and cannot be performed on a continuous basis. Also, a heat-dried preparation may be better suited than a freeze dried preparation for providing accurate quantitative results.

Suitable substrates typically include a solid support material. Solid support materials can include particulate materials, membranes, gels (e.g., agarose), or other solid support materials such as the surfaces of tubes, slides, or plates. Exemplary solid supports can include materials such as ferromagnetic materials, gold sols, silica, glass, and polymeric materials such as, for example, nitrocellulose, polystyrene, polypropylene, polycarbonate, and nylon. For certain embodiments, the substrate is particulate material (e.g., polystyrene beads having an average particle size of less than 1 micron (μm) such as, for example, approximately 0.3 μm). In certain embodiments, a particulate substrate may include paramagnetic articles or superparamagnetic particles.

The dry preparation also includes a stabilizer that is capable of stabilizing the biological material. Suitable stabilizers cause biological material to retain the enzymatic and chemical susceptibilities and recognition elements of living cells even though the biological material is rendered non-viable. In other words, a stabilizer prolongs the time during which the biological material can remain useful as, for example, a component of a positive control for a detection assay even after the biological material is no longer living.

Suitable stabilizers include materials that can help maintain the chemical integrity (e.g., protein conformation) and, therefore, biological functionality (e.g., affinity, enzymatic reactivity, etc.) of the biological material. Exemplary materials include, for example, polysaccharides (including, e.g., pectin and dextran), proteins (including, e.g., gelatin), and commercially available immunoassay stabilizers such as, for example, STABILCOAT (SurModics, Inc. Eden Prairie, Minn.). Certain stabilizers such as STABILCOAT can provide the additional benefit of serving as a blocking reagent in performing a detection assay.

In another aspect, the invention provides a method of making a dried biological preparation. Generally, the method includes collecting a sample of biological material, inactivating the biological material, suspending the sample in a volume of a stabilizer, and allowing the stabilizer to dry.

The biological sample may be collected in any suitable manner. In some cases, the sample may be collected from a solid, semi-solid, or broth cell culture. In other cases, the biological sample may be collected from a tissue (e.g., a biopsy) or physiological fluid (e.g., urine, saliva, mucus, blood, etc.) of a subject known to contain or suspected of containing the desired biological material.

In another aspect, the invention provides a method of performing a positive control in an assay for detecting target biological material. Generally, the method includes providing a dry preparation that comprises a stabilizer and stabilized, inactivated target biological material; contacting the inactivated target biological material with an aqueous solution, thereby generating a reconstituted sample; and performing the assay for detecting the target biological material on the reconstituted sample.

The particular methodology for performing the positive control is determined by the particular detection assay being performed. The use of a stabilized, inactivated target biological material in a positive control of a detection assay is compatible with virtually any known detection assay.

In certain embodiments, the assay can include lysing cells in the test sample and the positive control. The lysing may be performed by contacting the cells with a lysing agent. A suitable lysing agent may be, for example, an enzyme (e.g., a protease, a glycosidase, or a nuclease). Exemplary enzymes include lysostaphin, pepsin, glucosidase, galactosidase, lysozyme, achromopeptidase, endopeptidases, N-acetylmuramyl-L-alanine amidase, endo-beta-N-acethylglucosaminidase, ALE-1, DNase, and RNase. Various combinations of enzymes can be used if desired. Lysostaphin is particularly useful in assays for detecting the presence of Staphylococcus aureus.

Other lysing agents include salts (e.g., chaotrophic salts), solubilizing agents (e.g., detergents), reducing agents (e.g., beta-mercaptoethanol (BME), dithiothreitol (DTT), dithioerythritol (DTE), cysteine, n-acetyl cysteine) acids (e.g., HCl), and bases (e.g., NaOH). Various combinations of such lysing agents can be used if desired.

Analytical techniques employed in detection assays can be any of a wide variety of conventional techniques known to one of skill in the art. For example, such methods can include the use of fluorometric immunochromatography (e.g., rapid analyte measurement procedure such as that described in U.S. Pat. No. 5,753,517), acoustic wave sensors, ELISA (e.g., colorimetric ELISA), and other colorimetric techniques (e.g., colorimetric sensors including polydiacetylene (PDA) materials), as well as surface plasmon resonance (SPR, using biosensors of the type available from Biacore, Upsala, Sweden). In some embodiments, the detection assay can include the use of a immunochromatographic device such as, for example, a lateral flow device.

### EXAMPLES

The following examples have been selected merely to further illustrate features, advantages, and other details of the invention. It is to be expressly understood, however, that while the examples serve this purpose, the particular materials and amounts used as well as other conditions and details are not to be construed in a matter that would unduly limit the scope of this invention.

### Example 1

### Preparation and Coating Inactivated Staphylococcus aureus Cells Onto a Substrate

### A. Preparation of Inactivated Cells.

Staphylococcus aureus ATCC™ 25923 was obtained from the American Type Culture Collection (Manassas, Va.). Twenty milliliters (ml) of Tryptic Soy Broth (TSB) was inoculated with S. aureus 25923 and incubated at 37° C.±1° C. for approximately 21 hours. After incubation, an aliquot (1000 μl) of the broth culture was transferred into a 2 ml sterile centrifuge tube, in which it was washed twice with phosphate buffered saline (10 mM Na₂PO₄, pH 7.5, 150 mM NaCl) with 0.05% Tween 20 (PBS/Tween 20) using centrifugation at 12,847 RCF for 10 minutes at 4° C. to collect the cells. The final pellet was suspended in 1000 μl PBS/Tween 20. The washed cells were serially diluted in PBS/Tween 20 and 100 μl was spread in duplicate onto 5% sheep blood agar (SBA) using a sterile plastic spreader. The SBA plates were incubated at 37° C. overnight. After incubation, the dilution plates were counted and the S. aureus concentration calculated. The remainder of the broth culture was heat inactivated.

Immediately following the plating for enumeration, the remaining broth culture was transferred to a 50 ml conical centrifuge tube and heat inactivated by immersing the tube in a vigorously boiling water bath for 12 minutes±1 minute. The heat-inactivated broth culture was allowed to cool to room temperature. Ten 100 μl aliquots of the cooled, heat-treated bacterial suspension were transferred to ten individual tubes containing 1 ml TSB. These tubes were incubated at 37° C.±1° C. for at least 24 hours to verify that the S. aureus cells were no longer viable.

### B. Coating Inactivated Cells Onto a Substrate.

After the inactivated cells cooled to room temperature, the cells are washed twice with PBS/Tween 20 as described above. The final cell pellet was resuspended in 25% (v/v) STABILCOAT (SurModics, Inc., Eden Prairie, Minn.) in sterile reagent water. The resulting suspension was refrigerated overnight. This suspension was serially diluted in 25% STABILCOAT to achieve the desired concentration of heat-inactivated cells.

A 5 μl aliquot of the diluted cell suspension was spotted onto the inner surface of the bottom portion (sheath) of a sample tube (Medical Packaging Corporation, Camarillo, Calif.). The sheath(s) were placed in a convection oven at 40° C. until the coating solution has tactilely dried (approximately three hours). The dried sheaths immediately were removed from the oven, placed in a sealed in a foil pouch with desiccant, and refrigerated.

### Example 2

### Stability of Coated, Inactivated Cells in an ELISA Assay

### A. ELISA Procedure

An ELISA procedure was used to measure the binding activity of two protein antigens (Protein A and clumping factor) in the heat-inactivated S. aureus cells. In this procedure, unless specified otherwise, all of the wash procedures were performed using an automated plate washer that delivered five successive 200-μL aliquots of the PBS/Tween 20 (Example 1) wash solution. All incubations were carried out at 37° C., unless specified otherwise. Ninety-six well microtiter plates were obtained from Corning Inc. Life Sciences, Acton, Mass. All antibodies were biotinylated using an EZ-LINK NHS-PEO₄-Biotin kit (Pierce Biotechnology, Rockford, Ill.) according to the manufacturer's directions.

A 96-well plate was coated with primary antibody mixture containing 1 μg/ml of a monoclonal anti-Protein A (Mab 76, described in U.S. patent application Ser. No. 11/562,759), and 7.5 μg/ml of a monoclonal anti-clumping factor (12-9, U.S. Pat. No. 6,979,446). The plate was incubated for 1 hour and subsequently washed. STABILCOAT (200 μl/well) was added and the plate was refrigerated at 4° C. up to one week. The plate was washed prior to adding samples.

The plate was loaded with 100 μl/well of sample and incubated for 1 hour, then washed. A mixture of biotinylated antibodies, containing 2.5 μg/ml of monoclonal anti-Protein A antibody (Mab 107, described in U.S. patent application Ser. No. 11/562,747) and 0.75 μg/ml of affinity-purified polyclonal anti-clumping factor antibody (described in U.S. Provisional Patent Application Ser. No. 60/867,089), was added to the wells and the plate was incubated for 1 hour. After washing the plate, 100 μl/well (0.5 μg/ml) Streptavidin Alkaline Phosphatase (Jackson ImmunoResearch Laboratories, Inc., West Grove, Pa.) was added to each well. The plate was incubated for 1 hour and then washed. For color development, 100 μl of p-Nitrophenylphosphate phosphate substrate (KPL, Inc., Gaithersburg, Md.) was added to each well and the plate was allowed to stand at room temperature for 15 minutes. The enzyme reaction was stopped by adding 100 μl of EDTA (5% wt/vol) to each well. The enzyme activity was quantitated by measuring light absorption (405 nm) in a plate reader.

### B. Sample Processing

Experimental sample tubes that were prepared according to Example 1B were used in the following process steps: 1) the cap was removed from the sample tube and 360 μL of solution collected and pooled from several sterile processed sample acquisition devices was added to the sheath, 2) the sheath was vortexed for 30 seconds to resuspend the coated, inactivated cells, 3) 10 μl of 150 μg/mL lysostaphin (Sigma Chemical Co., St. Louis, Mo.) was added to the sheath, and 4) after mixing for 30 seconds, 100 μl of the suspension (called “sample” in Example 2A) was transferred to the microtiter plate for the ELISA assay.

“Control” samples were used in the stability studies described below. In these samples, the washed, diluted heat-inactivated cell suspensions were added to empty samples tubes, rather than using sample tubes in which the same suspensions were dried and stored. The control cell suspensions were prepared and stored identically to the material that was coated and dried in the sample tubes. Thus, the controls were processed as described above except that a 5 μl aliquot of the corresponding inactivated cell suspension was added to an empty sample tube in step 1 of the process described above.

### C. Storage Stability of the Coated, Dried, Heat-Inactivated Bacteria

The storage stability of the heat-inactivated S. aureus preparations was measured in the ELISA assay described in Example 2A. The objective was to determine whether the dried, heat-inactivated cells remain suceptible to lysostaphin and whether they retain the immunologic reactivity of certain antigens. Three different cell-suspending media were examined: phosphate buffered saline (10 mM Na₂PO₄, pH 7.5, 150 mM NaCl), 25% (v/v) STABILCOAT, and 100% STABILCOAT.

Samples of inactivated bacterial cells coated and dried in plastic sheaths, and their corresponding aqueous cell suspensions, were stored at three temperatures (−20° C. (freezer), 4° C. (refrigerator), and approximately 23° C. (room temperature)). Samples were removed periodically, processed according to Example 2B, and their antigenic reactivity was measured according to Example 2A. The results are shown in Table 1.

TABLE 1
Storage stability of dried, heat-inactivated S. aureus cells.

| | Timepoint and Storage Condition | | | | | | | | | | | |
|---|---|---|---|---|---|---|---|---|---|---|---|---|
| | | Day 1 | | Day 7 | | Day 14 | | Day 28 | | | Std | |
| | Day 0 | R | F | R | F | R | F | R | F | Mean | Dev | % CV |
| PBS Dried | 0.286 | 0.216 | 0.296 | 0.160 | 0.156 | na | na | na | na | 0.22 | 0.07 | 29.91 |
| PBS Control | 0.507 | 0.356 | 0.362 | 0.228 | 0.243 | na | na | na | na | 0.34 | 0.11 | 33.15 |
| 25% SC Dried | 0.839 | 0.731 | 0.679 | 0.941 | 0.861 | 1.104 | 1.226 | 1.341 | 1.226 | 0.98 | 0.22 | 12.00 |
| 25% SC Control | 0.868 | 1.029 | 0.906 | 0.893 | 0.995 | 1.030 | 0.776 | 1.068 | 0.776 | 0.94 | 0.09 | 9.80 |
| 100% SC Dried | 0.769 | 0.692 | 0.714 | 0.869 | 0.907 | 0.992 | 1.066 | 1.286 | 1.066 | 0.93 | 0.19 | 20.68 |
| 100% SC Control | 0.843 | 0.844 | 0.887 | 0.850 | 0.893 | 1.000 | .963 | 1.038 | 0.963 | 0.94 | 0.10 | 11.00 |

This table shows the absorbance readings from an ELISA assay detecting Protein A and clumping factor antigens. R = refrigerator storage, F = Freezer storage, na = not assayed. Day 0 was the day that the cell suspensions were inactivated.

The data indicate that heat-inactivated S. aureus cells stored in 100% STABILCOAT and 25% STABILCOAT at or below refrigerated storage temperature, both before and after coating and drying, remain susceptible to lysostaphin.

The complete disclosures of the patents, patent documents and publications cited herein are incorporated by reference in their entirety as if each were individually incorporated. In case of conflict, the present specification, including definitions, shall control.

Various modifications and alterations to this invention will become apparent to those skilled in the art without departing from the scope and spirit of this invention. Illustrative embodiments and examples are provided as examples only and are not intended to limit the scope of the present invention. The scope of the invention is limited only by the claims set forth as follows.

What is claimed is:

1. A biological sample comprising:
a dry preparation comprising a stabilizer and stabilized, inactivated biological material.

2. The biological sample of claim 1 wherein the biological material comprises at least one cellular component comprising a whole cell or at least one fragment of a whole cell.

3. The biological sample of claim 1 wherein the biological material comprises at least one viral component comprising a virus particle or a least one fragment of a virus particle.

4. The biological sample of claim 1 wherein the biological material is substantially homogeneous.

5. The biological sample of claim 1 wherein the material comprises a heterogeneous mixture of components.

6. The biological sample of claim 5 wherein the heterogeneous mixture comprises components from different organisms.

7. The biological sample of claim 5 wherein the heterogeneous mixture comprises components from a single organism.

8. The biological sample of claim 5 wherein the heterogeneous mixture comprises components from Staphylococcus aureus.

9. The biological sample of claim 8 wherein one component comprises an intracellular component and a second component comprises a cell surface component.

10. The biological sample of claim 1 wherein the dry preparation comprises a powder or tablet.

11. The biological sample of claim 1 further comprising a substrate having at least one coatable surface wherein the dry preparation comprises a coating disposed on at least a portion of the coatable surface.

12. The biological sample of claim 11 wherein the substrate comprises a plate, a slide, a tube, a particle, a membrane, or filter paper.

13. The biological sample of claim 11 wherein the dry preparation comprises a polysaccharide, a protein, or an immunoassay stabilizer.

14. The biological sample of claim 13 wherein the dry preparation comprises a material that stabilizes components of the sample.

15. The biological sample of claim 13 wherein the dry preparation comprises a blocking reagent.

16. The biological sample of claim 1 wherein the dry preparation is heat dried.

17. A method of making a dried biological preparation, the method comprising:
collecting a sample of biological material;
inactivating the biological material;
suspending the sample in a volume of a stabilizer; and
allowing the stabilizer to dry.

18. The method of claim 17 wherein the biological material comprises whole cells, cells fragments, virus particles, or fragments of virus particles.

19. The method of claim 17 wherein the biological material comprises at least one cellular component comprising a whole cell or at least one fragment of a whole cell.

20. The method of claim 17 wherein the biological material comprises at least one viral component comprising a virus particle or a least one fragment of a virus particle.

21. The method of claim 17 wherein the biological material is substantially homogeneous.

22. The method of claim 17 wherein the material comprises a heterogeneous mixture of components.

23. The method of claim 22 wherein the heterogeneous mixture comprises components from different organisms.

24. The method of claim 22 wherein the heterogeneous mixture comprises components from a single organism.

25. The method of claim 22 wherein the heterogeneous mixture comprises components from Staphylococcus aureus.

26. The method of claim 25 wherein one component comprises an intracellular component and a second component comprises a cell surface component.

27. The method of claim 17 further comprising disposing the biological material and stabilizing preparation combination on a substrate having at least one coatable surface.

28. The method of claim 27 wherein the substrate comprises a plate, a slide, a tube, a particle, a membrane, or filter paper.

29. The method of claim 27 wherein the stabilizing preparation comprises a polysaccharide, a protein, or an immunoassay stabilizer.

30. The method of claim 29 wherein the stabilizing preparation comprises a blocking reagent.

31. The method of claim 17 wherein inactivating the biological material comprises applying heat to the sample.

32. The method of claim 31 wherein inactivating the biological samples comprises transferring the sample to a vessel and heating the sample in boiling water for at least 12 minutes.

33. The method of claim 17 wherein inactivating the biological material comprises limitedly autoclaving the sample.

34. The method of claim 17 wherein inactivating the biological material comprises exposing the sample to UV irradiation.

35. The method of claim 17 wherein inactivating the biological material comprises chemically treating the sample.

36. The method of claim 35 wherein chemically treating the sample comprises treating the sample with azide or a metal.

37. The method of claim 35 wherein chemical treating the sample comprises enzymatically treating the sample or limitedly treating the sample with an antibiotic.

38. The method of claim 17 wherein inactivating the biological material comprises treating the sample with pressure.

39. The method of claim 17 wherein inactivating the biological material comprises mechanically treating the sample.

40. The method of claim 17 wherein inactivating the biological material comprises ultrasonically treating the sample.

41. The method of claim 17 wherein allowing the stabilizing preparation to dry comprises heat drying.

42. A method of performing a positive control in an assay for detecting target biological material, the method comprising:
providing a dry preparation that comprises a stabilizer and stabilized, inactivated target biological material;
contacting the inactivated target biological material with an aqueous solution, thereby generating a reconstituted sample; and
performing the assay for detecting the target biological material on the reconstituted sample.

43. The method of claim 42 wherein the target biological material comprises whole cells, cells fragments, virus particles, or fragments of virus particles.

44. The method of claim 42 wherein the biological material comprises at least one cellular component comprising a whole cell or at least one fragment of a whole cell.

45. The method of claim 42 wherein the biological material comprises at least one viral component comprising a virus particle or a least one fragment of a virus particle.

46. The method of claim 42 wherein the biological material is substantially homogeneous.

47. The method of claim 42 wherein the material comprises a heterogeneous mixture of components.

48. The method of claim 47 wherein the heterogeneous mixture comprises components from different organisms.

49. The method of claim 47 wherein the heterogeneous mixture comprises components from a single organism.

50. The method of claim 47 wherein the heterogeneous mixture comprises components from Staphylococcus aureus.

51. The method of claim 50 wherein one component comprises an intracellular component and a second component comprises a cell surface component.

52. The method of claim 42 wherein the assay comprises an ELISA or fluorometric immunochromatography.

53. The method of claim 52 wherein the assay includes using a lateral flow device.

54. The method of claim 42 wherein the target biological material comprises Staphylococcus aureus whole cells.

55. The method of claim 54 wherein the assay comprises lysing the Staphylococcus aureus whole cells and detecting an intracellular component of the Staphylococcus aureus.

56. The method of claim 55 wherein the Staphylococcus aureus whole cells are lysed with lysostaphin.
